(19) United States
(12) Patent Application Publication
(10) Pub. No.: US 2002/0009473 A1
(43) Pub. Date: Jan. 24, 2002

(54) Microcapsule, method for its production, use of same, and coating liquid with such

(76) Inventor: Gerold Tebbe, Monte Carlo (MC)

Correspondence Address: Jovan N. Jovanovic, Suite 5G/H, 1327 West Washington, Chicago, IL, 60607, US

(21) Appl. No.: 09/907,294

(22) Filed: Jul. 17, 2001

(30) Foreign Application Priority Data
Jul. 18, 2000 (DE) 10035302.9

(51) Int. Cl. A61K 9/48; A61K 7/00

(52) U.S. Cl. 424/401; 424/490

(57) ABSTRACT

Thermostatting microcapsules (10) have a core (12) which consists of a material that has a melting point in the range from 5° C. to 40° C. The core is surrounded by a first wall (14) which is obtained by the spraying around of a core drop. The wall (14) may be incomplete, so that core material is still incorporated in it. The first wall (14) is surrounded by a further wall (16) which has been produced in a separate method step, namely by a fluidized bed process. The core material (12) is enclosed reliably and tightly in the microcapsule (10) so obtained.

The invention relates to a microcapsule according to the preamble of claim 1, a method for its production, its use, and a coating liquid suitable for applying such microcapsules.

It is known that materials in the cavity (core materials) of microcapsules may be used for thermostatting purposes by melting and solidification, since they withdraw heat from the environment during the melting and release it to the environment during the solidification. Paraffin oil and wax is particularly preferred for this purpose because of its chemical inertness and its non-toxicity.

Other core materials may be skin-care materials such as skin oil or skin cream.

The core materials are conventionally microencapsulated with the aid of a suitable film-forming polymer. To date microcapsules which comprise e.g. paraffin oil or wax as thermostatting core material and a film-forming polymer, such as for example acrylic resin, as wall material have been produced in such a way that the film-forming polymer and the paraffin oil or wax have been atomized simultaneously in a nozzle.

A disadvantage of said production method consists in the fact that the paraffins or the skin-care material are covered incompletely by the wall material during the atomizing, so that they are incorporated partially into the wall material. The capsules thereby become unstable, since the paraffin oil or wax, skin oil or skin cream contained in the wall material may leak out of the wall material in particular at higher temperatures or the capsule may even disintegrate completely.

The object of the invention is to create a microcapsule of encapsulated core material, in particular paraffin oil and paraffin wax or skin oil or skin cream, which is chemically and mechanically stable and may therefore be used as a thermostatting or skin-care agent.

This object is achieved by the microcapsule of claim 1.

The invention provides microcapsules which do not exhibit the disadvantages of the prior art, namely the presence of core material in the outer microcapsule wrapping. The advantage of the invention lies in the fact that there is arranged around a first wall layer of the microcapsule, which according to the method described above may contain inclusions of core material, a discretely formed further wall layer containing no core material and having a self-contained surface. The microcapsules therefore comprise a stable outer wrapping, whereby an acquirement of tackiness or an exuding of core material or a disintegration due to instability is prevented.

The development of the invention according to claim 2 covers microcapsules which may serve as heat-absorbing or heat-releasing particles.

Preferred thermostatting core materials of the present invention are paraffins, since, as already mentioned, they are chemically inert and non-toxic. Particularly preferred are the paraffins n-tetradecane (mp melting point 5.9° C.), n-pentadecane (mp 10.0° C.),n-hexadecane (mp 18.2° C.), n-heptadecane (mp 22.0° C.), n-octadecane (mp 28.2° C.), n-nonadecane (mp 32.1° C.) and n-icosane (mp 36.8° C.) (claim 3). Other organic core materials may also be used, however, such as 2,2-dimethyl-1,3-propane diol (DMP) or 2-hydroxymethyl-2-methyl-1,3-propane diol.

In general the melting points of the core materials of the microcapsules according to the invention lie preferably in the range from 5° C. to 40° C. A preferred melting point range of the core materials lies between 20° C. and 40° C. (claim 4).

Microcapsules such as are given in claim 5 may be used as a skin-care coating.

With the development of the invention according to claim 6 it is brought about that the microcapsules may in an early finishing step be combined with a material without their being damaged in further finishing steps. In the last finishing step or a separate handling step, the outermost wall layer of the microcapsules is then removed, so that their contents may then be released with destruction of the remaining capsule wall now exposed, e.g. by the scouring of said capsule wall.

The wall material of the microcapsules consists preferably of film-forming polymer (claim 7).

The materials for the first and second walls may be different (claim 8) or else identical (claim 9).

Preferred wall materials are acrylic resins, polyethylene, polypropylene, ethylene-propylene-copolymer, ethylenepropylene-diene-terpolymer, acrylates and methacrylates, polyester, polystyrene and silicones (claim 10). Among said materials, acrylic resin is particularly preferred for both wall layers, since it supplies particularly stable walls which are able to withstand without difficulty mechanical loads such as rubbing against the skin on the inside of articles of clothing.

In claim 11 is described a particularly suitable method for producing the microcapsules according to the invention.

Sub-claims 12 to 14 are preferred developments of the method.

The preferred method for producing the microcapsules according to the invention comprises two stages: In a first stage, paraffin oil or liquid paraffin wax or another liquid or cream-like core material is atomized through a nozzle together with a solution of a wall material and optionally then dried. This may in particular be carried out in such a way that drops are formed by the pressing out of the core material from a core-forming nozzle and further the wall material is discharged out of a wall-forming nozzle arrangement adjoining the core-forming nozzle in such a way that the wall material places itself around the core drop and forms a wall. The wall layer downstream of the first stage is however not completely self-contained, but also contains core material. Thereafter the microcapsule intermediate products of the invention may be cooled to below the solidifying point of the core material for the purpose of stabilization during storage and further processing (claim 15).

In the second stage of the method the microcapsule intermediate products obtained in the first stage are sprayed in the flowing state in a fluidized bed process at the lowest possible temperature (in order that the microcapsule intermediate products are not destroyed) with a solution of a wall material the same as or different from that in the first stage.

Preferably a vacuum fluidized bed process is used, in the vacuum of which the solvent is evaporated from the surface of the microcapsules and leaves behind a continuous film of wall material. Fluidized bed processes of this kind are known per se in engineering. For example, there is suitable for the second stage of the present invention a vacuum fluidized bed process having a solvent recovery system, such as is used, for example, for the atomizing of retard films on pharmaceutical pills. An example of a commercially available vacuum fluidized bed unit of this kind is the Glatt-R-vacuum fluidized bed unit.

If the microcapsules are not yet completely free of solvent at the end of the second method stage, they are dried in a separate drying step (claim 13).

The second stage of the method according to the invention supplies microcapsules which comprise a stable outer wall layer containing no core material and which are heat resistant and also highly stable mechanically.

For this reason the microcapsules are also suitable for being applied to the inside of articles of clothing (claim 16). The friction with the body is not sufficient to destroy the microcapsules, and thus they may achieve a thermostatting effect on the body.

There is achieved with the development of the invention according to claim 17 a simple and reliable production of the coating on the inside of an article of clothing.

Further, according to claim 18 an article of clothing may be finished as a whole in such a way that it both has a thermostatting effect and ensures a skin-care effect.

With the development according to claim 19 it is brought about that despite the coating an air permeability of the coated article of clothing is created.

Screen sizes for the printing on of the coating, such as are specified in claim 20, may be easily achieved. Nevertheless the printed-on coating has substantially a homogeneous effect on the skin.

Denim trousers are a particularly preferred article of clothing for said use of the microcapsules according to the invention (claim 21).

According to claim 22 a washable article of clothing may be given a thermostatting or skin-care finish.

Claim 23 specifies preferred binders with which microcapsules may be combined with an article of clothing. Examples are: polyurethane, types of nitrile rubber, types of chloroprene rubber, polyvinyl alcohol, ethylene-vinyl acetate copolymers, acrylic resins, silicones, e.g. silicone elastomers, starch and cellulose. Silicone elastomers are particularly preferred.

The thermostatting effect is reinforced if a mixture of microcapsules is used in which each of the components of the mixture comprises a paraffin oil or wax with a particular melting point within the range of from 5° C. to 40° C. and the melting points of the paraffin components are distributed over the whole range from 5° C. to 40° C. (claim 24).

Rates of application of the coating such as are given in claim 25 ensure on the one hand that the desired thermostatting and/or skin-care effect is also retained over a protracted period, and on the other hand that the handle of the textile material is influenced not insignificantly by the coating.

A coating liquid which contains microcapsules of the invention, a binder and optionally a solvent and/or a propellant and may be employed for the use according to the invention is given in claim 26. By means of it a user may himself give an article of clothing a thermostatting and/or skin-care finish.

Short Description of the Invention

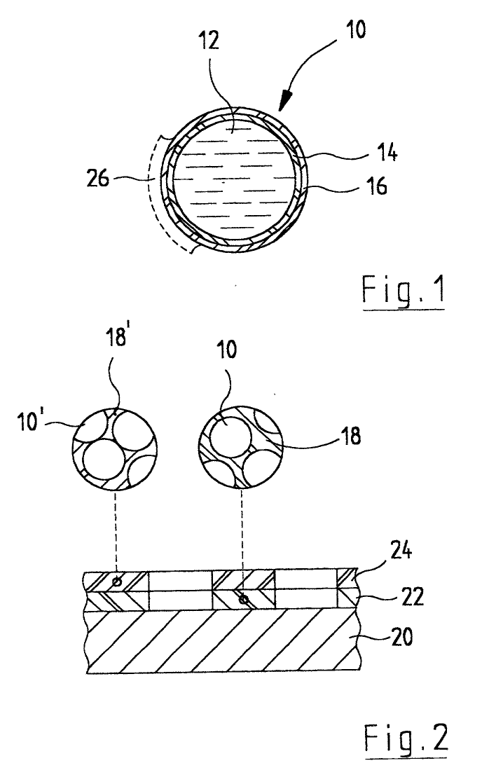

FIG. 1 is a diagrammatic section through a microcapsule according to the invention;

FIG. 2 is a section through an article of clothing which is provided with a lower thermostatting coating and an upper skin-care coating.

A core 12 of a microcapsule 10 is formed of a core material, e.g. paraffin oil or wax, with a melting point of between 5° C. and 40° C. The core 12 is surrounded by a first wall layer 14 which is formed conventionally of a film-forming polymer, which envelopes the core 12, but may also contain included core material. The first wall layer 14 is surrounded by a further wall layer 16 which contains no core material and has a self-contained structure on the lines of a spherical shell.

The microcapsule 10 is represented diagrammatically as a sphere, but may in practice also assume many other forms, wherein the diameters may lie in a range from 0.05 μm to about 1000 μm.

As a modification of the embodiment described above it is also possible to produce microcapsules in which the core 12 is formed by a skin-care material, e.g. a skin oil or a skin cream.

The skin-care agent is incorporated tightly and reliably on the inside of the two wall layers 14, 16. The contents of the microcapsule are released by mechanical destruction of the two wall layers 14, 16. Such a mechanical destruction takes place e.g. if such microcapsules are embedded in a binder and the mixture so obtained is applied to an article of clothing which is then worn in frictional contact with the surface of the skin. The microcapsules are then successively “ground” there by the friction on the skin until access to the core 12 exists. The core material is then released slowly and is available for the care of the skin over a long period, since the grinding down of the various microcapsules takes place at different points in time.

In FIG. 2 the symbol 20 denotes an article of clothing. The latter bears a lower thermostatting coating 22, which is applied by half-tone screen printing. The screen size is chosen as between 0.5 to 2 mm in practice. Above the thermostatting coating 22 is provided true to register a skin-care coating 24. The latter is also produced by half-tone screen printing.

The material-filled areas of the two coatings 22, 24 and the sections of the coatings 22, 24 not occupied by material are therefore in alignment with one another, so that the whole of the article of clothing 20 bearing the two coatings 22, 24 remains air permeable.

As shown in an enlargement, the coatings 22, 24 each include a binder 18 or 18′, in which microcapsules 10 or 10′ are accommodated.

The binder is an organic plastic and preferably selected from the following group: polyurethane, types of nitrile rubber, types of chloroprene rubber, polyvinyl alcohol, ethylene-vinyl acetate copolymers, acrylic resin, silicones, e.g. silicone elastomers, starch and cellulose. Preferably a silicone elastomer or a comparable abrasion-resistant plastic is used.

The coatings 22, 24 each have a weight per unit area of 0.5 to 200 g/m², preferably 1 to 60 g/m².

As a modification of the above embodiment it is also possible to use a binder which produces a porous binder layer. Such a binder may then be applied over the whole surface by spraying or knife coating.

If an article of clothing such as is shown in FIG. 2 is worn, the skin-care coating 24 is in constant frictional contact with the surface of the skin. The binder 18′ and the wall material of the microcapsules 10′ are worn away by the frictional contact, and the contents of the micro-capsules 10′ are progressively released. A skin-care effect is thereby obtained.

The thermostatting coating is not changed during normal wearing of the article of clothing.

The binder 18 is so selected that it withstands a fairly large number of washings (about 10).

As a modification of the above embodiment there may be provided in particular for capsules containing a skin-care agent a further wall, such as that shown at 26 in FIG. 1.

The wall 26 is produced from a material which is resistant to the substances or handling liquids which are used in the finishing steps to which a material is subjected, optionally with the exception of the last finishing step. It is therefore possible for said capsules to be applied to the material at an early stage in the finishing of the latter, which is advantageous in terms of a firm adhesion. Conversely a desirably destructible wall of the capsules is exposed again before the product is delivered to the end consumer. This may take place either in the last finishing step provided in any case, or in an additional working step in which an agent attacking the material of the outermost wall is employed.

1. A microcapsule having a core (12), and having a first wall layer (14) surrounding the core (12), wherein the first wall layer (14) is surrounded by at least one further wall layer (16) containing no core material.

2. A microcapsule as claimed in claim 1, wherein the core material has a melting point of between about 5° C. and about 40° C.

3. A microcapsule as claimed in claim 2, wherein the core (12) comprises a paraffin oil or a paraffin wax, e.g. n-tetradecane, n-pentadecane, n-hexadecane, n-heptadecane, n-octadecane, n-nonadecane and n-icosane.

4. A microcapsule as claimed in claim 2 or 3, wherein the core material has a melting point of between about 20 and about 40° C.

5. A microcapsule as claimed in claim 1, wherein the core (12) is a skin-care agent, in particular a skin-care oil or a skin cream.

6. A microcapsule as claimed in one of claims 1 to 5, wherein it comprises an outer wall (26) which is resistant to substances such as are used in finishing steps of a material, optionally with the exception of the last finishing step.

7. A microcapsule as claimed in one of claims 1 to 6, wherein the first wall layer (14) surrounding the core (12) and the further wall layer (16) are formed by a film-forming polymer.

8. A microcapsule as claimed in claim 7, wherein the first wall layer (14) surrounding the core and the further wall layer (16) are formed by two film-forming polymers which differ in their chemical and/or physical and/or mechanical properties.

9. A microcapsule as claimed in claim 7, wherein the first wall layer (14) surrounding the core and the further wall layer (16) are formed by the same film-forming polymer.

10. A microcapsule as claimed in one of claims 7 to 9, wherein the film-forming polymer is selected from the following group: acrylic resins, polypropylene, polyethylene, acrylates and methacrylates, polyester, polystyrene and silicones.

11. A method for producing microcapsules as claimed in one of claims 1 to 10, in which in a first method step a core material which has a melting point of between about 5° C. and 40° C. is coated in a nozzle with a first wall material with the formation of a first wall layer (14), wherein in a further method step the microcapsules obtained in the first method step are coated in a fluidized bed process with a wall material with the formation of a wall layer (16) containing no core material.

12. A method as claimed in claim 11, wherein the first method step is carried out as follows:
a) The core material is pressed out to a drop from a core-forming nozzle;
b) the first wall material is discharged out of a wall-forming nozzle arrangement adjoining the core-forming nozzle in such a way that the wall material places itself around the core drop and forms a wall.

13. A method as claimed in one of claims 11 or 12, wherein the microcapsules obtained in the further method stage and comprising core and wall layers are dried prior to a further use.

14. A method as claimed in one of claims 11 to 13, wherein the microcapsules obtained in the first method stage are pre-dried prior to the further method stage.

15. A method as claimed in one of claims 11 to 14, wherein the microcapsules obtained in the first method stage are cooled down prior to the further method stage to a temperature lying below the solidifying temperature of the core material.

16. A use of microcapsules as claimed in one of claims 1 to 10 as a thermostatting or skin-care coating (22; 24) on the inside of an article of clothing (20).

17. A use as claimed in claim 16, wherein the microcapsules (10) are applied together with a binder (18) to the inside of the article of clothing (20), e.g. by spraying, knife coating or printing.

18. A use as claimed in claim 16 or 17, wherein there is applied to the inside of the article of clothing (20) first of all a thermostatting coating (22) and on top of the latter a skin-care coating (24).

19. A coating as claimed in one of claims 16 to 18, wherein the coating (22; 24) is applied by half-tone printing.

20. A use as claimed in claim 19, wherein the screen size amounts to 0.5 to 2 mm.

21. A use as claimed in one of claims 16 to 20, wherein the articles of clothing are denim trousers.

22. A use as claimed in one of claims 16 to 21, wherein a binder (18; 18′) of the coating (22; 24) is not water-soluble or dispersible in water.

23. A use as claimed in one of claims 16 to 22, wherein the binder (18; 18′ ) comprises an organic plastic, for example polyurethane, types of nitrile rubber, types of chloroprene rubber, polyvinyl alcohol, ethylene-vinyl acetate copolymers, acrylic resin, silicones, e.g. silicone elastomers, starch and cellulose.

24. A use as claimed in one of claims 16 to 23, wherein a mixture of microcapsules (10) is used, which contain different paraffin oils or waxes each of which is incorporated in a separate microcapsule.

25. A use as claimed in one of claims 16 to 24, wherein the thermostatting or skin-care coating (22; 24) is applied in an amount of 0.5 to 200 g/m², preferably in an amount of 1 to 60 g/m².

26. A coating liquid for a use as claimed in one of claims 16 to 25, wherein it contains, as defined in claims 1-10, microcapsules, a binder and optionally a solvent and/or a propellant.
